(19) United States
(12) Patent Application Publication
(10) Pub. No.: US 2008/0312719 A1
(43) Pub. Date: Dec. 18, 2008

(54) TRANSDUCER WIRELESS CONTROL SYSTEM AND METHOD

(75) Inventor: George Keilman, Bothell, WA (US)

Correspondence Address: DAVIS WRIGHT TREMAINE, LLP/Seattle, 1201 Third Avenue, Suite 2200, SEATTLE, WA, 98101-3045, US

(73) Assignee: CARDIOMETRIX, INC., Bothell, WA (US)

(21) Appl. No.: 12/030,829

(22) Filed: Feb. 13, 2008

Related U.S. Application Data
(60) Provisional application No. 60/943,799, filed on Jun. 13, 2007.

(51) Int. Cl. A61N 1/08

(52) U.S. Cl. 607/60

(57) ABSTRACT

A transducer wireless control system provides wireless control of transmit and receive activity of ultrasonic or other types of transducers used as sensors or other applications. In other applications, the transducer wireless control system provides wireless control of the phase of transmitted and received signals to or from ultrasonic or other transducers. For instance, some versions of the transducer wireless control system have options to invert or not invert one or both of a pair of signals, thereby enabling addition and subtraction of RF waveforms.

### CROSS REFERENCE TO RELATED APPLICATION

This application claims priority benefit of provisional application Ser. No. 60/943,799 filed Jun. 13, 2007, the content of which is incorporated in its entirety.

### BACKGROUND OF THE INVENTION

1. Field of the Invention

The present invention is directed generally to wireless sensors.

2. Description of the Related Art

For sensing and measurement applications in environments such as inside of a human or an animal subject, it is helpful or even required to have component size be quite small, such as on the order of millimeters or less. It is also useful or required to have device control of the components utilize wireless methods. Conventional approaches often rely on application specific integrated circuit (ASIC) devices or similar approaches. Unfortunately, these conventional approaches can require component sizes and numbers too large for certain applications.

### BRIEF DESCRIPTION OF THE SEVERAL VIEWS OF THE DRAWING(S)

FIG. 1 is a schematic circuit diagram of a first implementation of a transducer wireless control system.

FIG. 2 is graphical representation of an exemplary RF signal used in the transducer wireless control system.

FIG. 3 is a schematic circuit diagram of a second implementation of the transducer wireless control system.

FIG. 4 is a schematic circuit diagram of a third implementation of the transducer wireless control system.

FIG. 5 is a schematic circuit diagram of a fourth implementation of the transducer wireless control system.

FIG. 6 is an exemplary graph showing resultant signals associated with both sum and difference versus phase difference.

### DETAILED DESCRIPTION OF THE INVENTION

A transducer wireless control system provides wireless interrogation and/or control of transmit and receive activity of ultrasonic or other types of transducers used as flow sensors or for various other applications. In some applications, the transducers are included in an implanted device placed in intravascular locations in animals or in the human body for the purpose of measuring blood flow, pressure, fluid attenuation, wall motion, or other physiologic parameters.

In other applications, the transducer wireless control system provides wireless control of the phase condition of transmitted and received signals to or from ultrasonic or other transducers. For instance, some versions of the transducer wireless control system have options to invert or not invert one or both of a pair of signals, such as ultrasonic signals, thereby enabling analog addition and subtraction of RF waveforms, which can be integral to a simple ultrasonic flow measurement scheme.

Implementations of the transducer wireless control system use a few basic electronic components that allow the implanted device to collapse to a size suitable for insertion into a typical intravascular or intracardiac catheter, cannula, or guidewire diameter or diameter of another tubular structure. The system can use a small number of tiny electronic components so can accommodate such applications as being included in an implant assembly that fits inside a catheter having a diameter on the order of 0.2 to 6 mm. As the electronic components must fit as a subassembly in the implant assembly, the size and number of components are kept to a minimum.

The transducer wireless control system uses components that are inherently robust to withstand large electrical transients that may be caused by medical systems such as an MRI scanner, cardiac defibrillators, or other devices.

The transducer wireless control system can include an electronic system, which is wirelessly coupled via an RF magnetic field to a transducer sub-system. In one application, the transducer sub-system can be implantable in a human or an animal subject for purposes of monitoring or controlling transducer sensors that are also implantable. One specific application is in the measurement of blood flow, blood pressure, ultrasonic attenuation within the blood (e.g., to measure viscosity, which has been shown to be proportional to hematocrit), vessel or cardiac wall motion or distension (e.g, as a function of internal pressure), and other physiological parameters from within a blood vessel or within the heart itself.

First Implementation

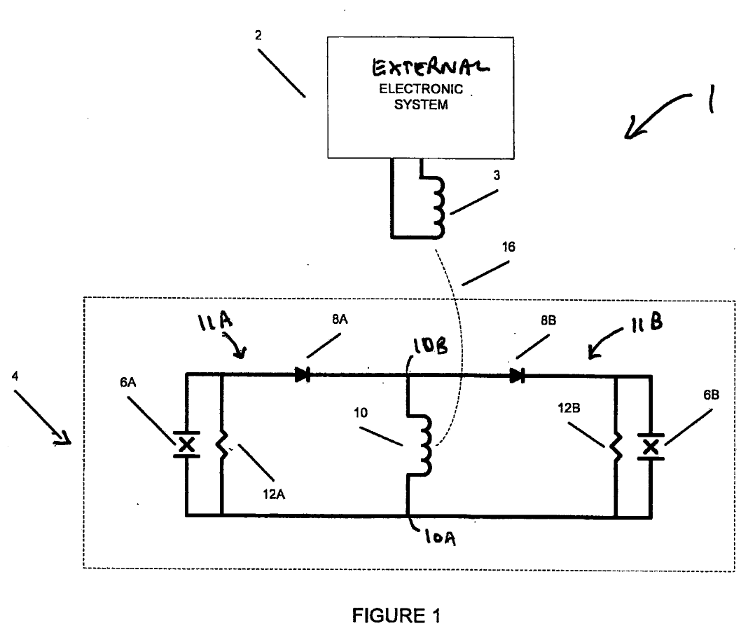

A first implementation of a transducer wireless control system 1 is shown in FIG. 1 to include an external electronic system 2 coupled via an inductive antenna 3 to a first transducer sub-system 4 via a second inductive antenna 10. “External” refers to the external electronic system 2 being physically separated from the first transducer sub-system 4. The first transducer sub-system 4 can typically be implanted inside of a body such as a human body whereas the external electronic system 2 can typically be located outside of the body or elsewhere. In the first implementation, the inductive antenna 10 has a first connection portion 10A and a second connection portion 10B and is connected in parallel between the first connection portion 10A and the second connection portion 10B to a first sub-circuit 11A having a diode 8A connected in series with a parallel combination of a transducer 6A and a resistor 12A. The inductive antenna 10 is also connected in parallel between the first connection portion 10A and the second connection portion 10B to a second sub-circuit 11B having a diode 8A connected in series to a parallel combination of a transducer 6B and a resistor 12B. The diode 8A and the diode 8B are connected to the inductive antenna 10 at the second connection portion 10B being oppositely polarized with respect to each other. The forward biased current flow for the first diode 8A goes from the first connector portion 10A through the first sub-circuit 11A through the first diode toward the second connector portion 10B. The forward biased current flow for the second diode 8B goes from the second connection portion 10B through the second diode through the sub-circuit 11B toward the first connection portion 10A.

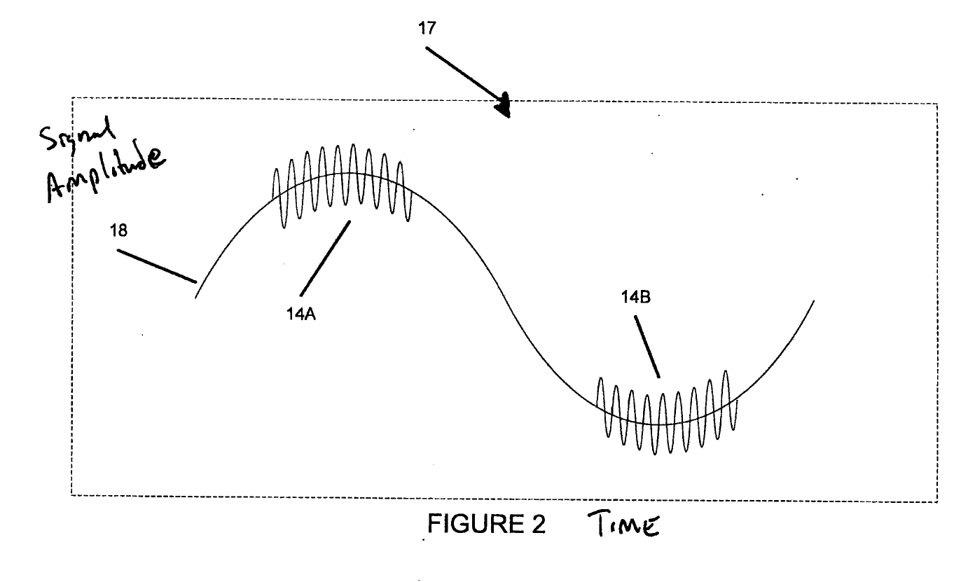

As shown in FIG. 1, the external electronic system 2 uses the inductive antenna coil 3 to generate an external magnetic field 16. The external magnetic field 16 has an amplitude-time waveform 17 shown in FIG. 2. The waveform 17 is a composite magnetic field having two components, a low frequency (hereafter ‘LF’) component 18 and one or more high frequency (hereafter ‘HF’) components. A first HF component 14A is depicted as being associated with a positive amplitude of the LF component 18 and a second HF component 14B is depicted as being associated with a negative amplitude of the LF component 18.

The LF component 18 of the external magnetic field 16 will have sufficient field strength to generate a voltage across the inductive antenna 10 that alternately forward biases the diode 8A and the diode 8B. When the diode 8A is forward biased, the first connection portion 10A is at a sufficiently positive voltage potential with respect to the second connection portion 10B and if an HF component exists, the external magnetic field 16 has amplitude that includes the HF component 14A. When the diode 8B is forward biased, the first connection portion 10A is at a sufficiently negative voltage potential with respect to the second connection portion 10B and if the external magnetic field 16 has an HF component, the external magnetic field will have amplitude that includes the HF component 14B.

Referring again to FIGS. 1 and 2, when the diode 8A is forward biased, the inductive antenna 10 is in series with the first sub-circuit 11A. Current generated by the HF component 14A at the inductive antenna 10 is conducted to the transducer 6A causing the transducer to emit energy at the frequency of the HF component.

Alternately, during the forward bias condition of the diode 8A, the external electronic system 2 can generate the external magnetic field 16 having only the LF component 18 and not the HF component 14A. As a result, any signal such as an ultrasonic signal having an HF component that impinges on the transducer 6A will cause the transducer to produce a current that will conduct to the inductive antenna 10 where an internally produced version of the HF component 14A can be detected by the external electronics 2 via the external antenna 3.

When the diode 8B is forward biased, the inductive antenna 10 is in series with the second sub-circuit 11B. Current generated by the HF component 14B at the inductive antenna 10 is conducted to the transducer 6B causing the transducer to emit energy at the frequency of the HF component.

Alternately, during the forward bias condition period of the diode 8B, the external electronic system 2 can generate the external magnetic field 16 having only the LF component 18 and not the HF component 14B. As a result, any signal such as an ultrasonic signal having an HF component that impinges on the transducer 6B will cause the transducer to produce a current that will conduct to the inductive antenna 10 where an internally produced version of the HF component 14B can be detected by the external electronics 2 via the external antenna 3.

The diode 8A and the diode 8B can be selected to have a high values for reverse breakdown voltage, so that large external magnetic field transients will not damage the transducer subsystem 4. Such external magnetic field transients may be produced by MRI systems, cardiac defibrillators (external or implanted), or other sources of environmental magnetic fields. The diode 8A and the diode 8B can be conventional PN junction diodes with switching times that are appropriate for the frequencies being used in the design. Alternately, the diode 8A and the diode 8B can be PIN diodes, i.e., diodes with an intrinsic silicon region separating their P and N-doped regions. When the diode 8A and the diode 8B are forward biased as PIN diodes, they will remain conductive for a carrier lifetime, which follows the forward bias period. Thus, the diode 8A and the diode 8B, as PIN diodes will continue to conduct for a brief period, immediately following the removal of a forward bias current. Consequently, use of PIN junction diodes for the diode 8A and the diode 8B may be advantageous in reducing power requirements to switch the diodes on and off. If PIN diodes are used, the reverse-biased diode may need to be reverse-biased for a longer time or with a larger bias voltage in order to fully shut it off.

In situations where the transducer 6A and the transducer 6B are not sufficiently conductive at the LF frequency, the resistor 12A and the resistor 12B are needed, respectively, to carry the bias current due to the LF component 18 to the diode 8A and the diode 8B. If the transducer 6A and the transducer 6B are sufficiently conductive to carry the LF current, then resistors 12A and 12B can have a high value or they can be removed entirely.

Second Implementation

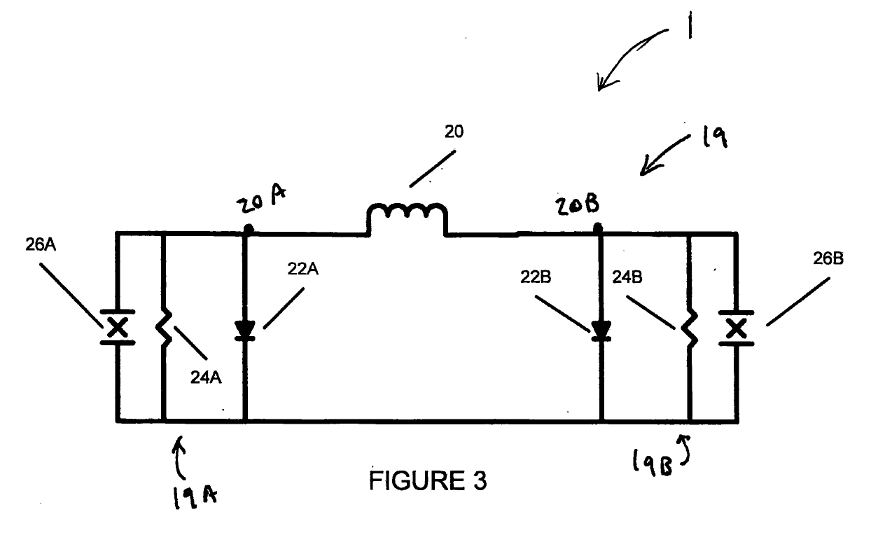

A second implementation of the transducer wireless control system 1 has a second transducer sub-system 19 shown in FIG. 3 as having a first sub-circuit 19A, a second sub-circuit 19B, and an inductive antenna 20. The antenna 20 is connected in series with the first sub-circuit 19A at a first connection portion 20A and is connected in series with the second sub-circuit 19B at a second connection portion 20B. The first sub-circuit 19A and the second sub-circuit 19B are connected together in series. The first sub-circuit 19A is shown to have a diode 22A, a resistor 24A, and a transducer 26A connected together in parallel. Like other resistors mentioned herein, the resistor 24A allows for current flow when current flow is not occurring through its associated diode. The second sub-circuit 19B is shown to have a diode 22B, a resistor 24B, and a transducer 26B connected together in parallel.

In the second implementation, the LF component 18 of the external magnetic field 16 is produced to have sufficient field strength to generate a voltage across antenna 20 that alternately forward biases the diode 22A and the diode 22B. When the voltage potential of the first connection portion 20A is sufficiently positive with respect to the second connection portion 20B, the diode 22A becomes forward biased and if the external magnetic field 16 has an HF component, it will include the HF component 14A, which will generate an HF voltage at the antenna 20.

When the diode 22A is forward biased, a circuit results that has the diode 22A connected in series with the antenna 20 and connected in series with effectively a portion of the second sub-circuit 19B having the resistor 24B connected with the transducer 26B in parallel. Forward biased diode 22A effectively shorts transducer 26A and resistor 24A. Reversed biased diode 22B presents high impedance so is effectively an open which can be disregarded in this instance regarding the second sub-circuit 19B. The HF component 14A of the external magnetic field 16 will generate current at the antenna 20 that will be conducted to the transducer 26B thereby causing the transducer to emit energy at the HF component frequency.

Alternately, during this forward bias condition of the diode 22A, the external electronic system 2 can be controlled to generate no HF component to the external magnetic field 16 from the external electronic system 2. Consequently, an HF frequency signal impinging on the transducer 26B will cause the transducer to produce a current which will conduct to the antenna 20 where it will internally produce the HF component of the external magnetic field 16 to be detected by the external electronic system 2.

When the diode 22B is forward biased, a circuit results that has the diode 22B connected in series with the antenna 20 and connected in series with effectively a portion of the first sub-circuit 19A having the resistor 24A connected with the transducer 26A in parallel. Forward biased diode 22B effectively shorts the transducer 26B and the resistor 24B. The reverse biased diode 22A presents high impedance so is effectively an open circuit condition which can be disregarded in this instance regarding the first sub-circuit 19A. The HF component 14B of the external magnetic field 16 will generate current at the antenna 20 that will be conducted to the transducer 26A thereby causing the transducer to emit energy at the HF component frequency.

Alternately, during this forward bias condition of the diode 22B, the external electronic system 2 can be controlled to generate no HF component to the external magnetic field 16 from the external electronic system 2. Consequently, an HF frequency signal impinging on the transducer 26A will cause the transducer to produce a current which will conduct to the antenna 20 where it will internally produce the HF component of the external magnetic field 16 to be detected by the external electronic system 2.

Third Implementation

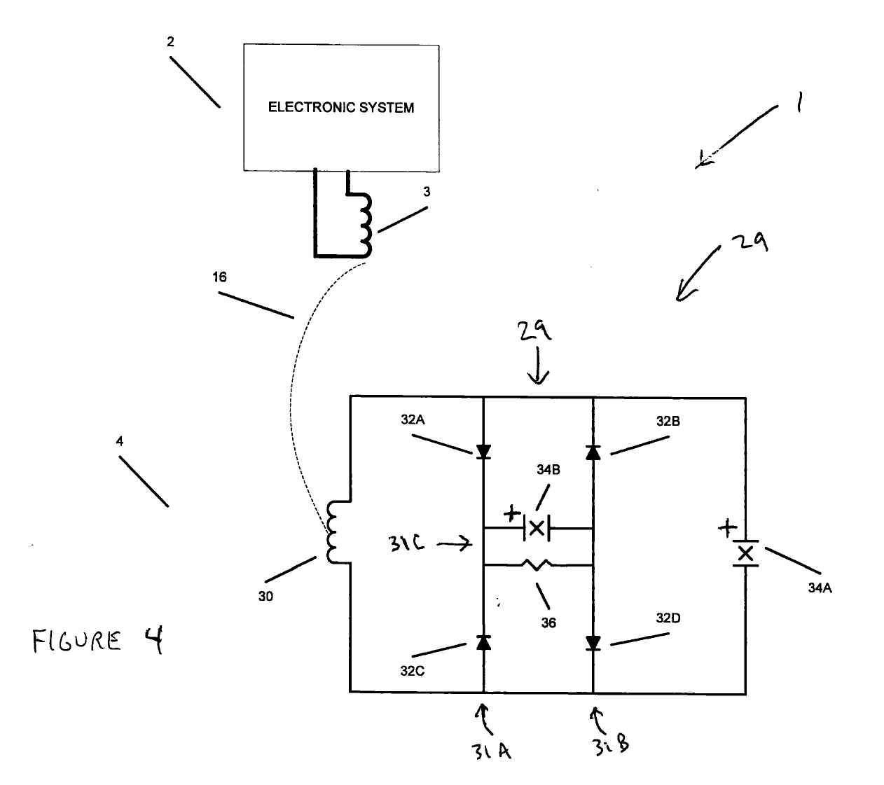

A third implementation of the transducer wireless control system 1 has a third transducer sub-system 29 shown in FIG. 4 as including an inductive antenna 30 connected in parallel with a sub-circuit 29A, and a transducer 34A. The sub-circuit 29A is depicted as a full-wave diode bridge network being a parallel connection of a first component portion 31A and a second component portion 31B. The first component portion 31A has a diode 32A and a diode 32C connected in series and oppositely polarized, with their cathodes connected with each other. The second component portion 31B has a diode 32B and a diode 32D connected in series and oppositely polarized, with their anodes connected with each other. The third component portion 31C has transducer 34B and a resistor 36 connected in parallel with each other, and is connected between the common cathode of component portion 31A and the common anode of component portion 31B. The arrangement of the third transducer sub-system 29 provides a selection of connecting the transducer 34B to the transducer 34A in parallel with the same or opposite polarity as dynamically selected.

The third transducer sub-system 29 enables the external electronic system 2 to control the transmit and receive polarity of the transducer 34A relative to the transducer 34B. The HF component 14A, the HF component 14B, and the LF component 18 are coupled to antenna 30 via the external magnetic field 16 similarly to that described regarding the external magnetic field and the first transducer sub-system 4. For the case of the third transducer sub-system 29, if the HF component 14A excites the antenna 30 the resultant HF voltage on the antenna is coupled directly to the transducer 34A.

When the LF component 18 produces a voltage on the antenna 30 sufficient to forward bias the diode 32A and the diode 32D a circuit is established with the antenna 30, the diode 32A, the diode 32D, the resistor 36, and the transducer 34B. Consequently, the HF voltage at antenna 30 caused by the HF component 14A will be coupled to the transducer 34B with an in-phase phase condition having the same phase as the transducer 34A.

Alternatively, when the diode 32A and the diode 32D are forward biased, the external electronic system 2 can be controlled to generate only the LF component 16 without the HF components 14A and 14B. Consequently, an HF frequency signal such as an ultrasonic signal impinging on the transducer 34B will cause the transducer 34B to produce a current which will add to any current from the transducer 34A caused by another HF frequency signal impinging upon the transducer 34A. The combined current will conduct to the antenna 30, where the HF component 14A will be internally produced to be detected by the external electronic system 2.

When the magnetic field 16 produces a voltage on the antenna 30 sufficient to forward bias the diode 32B and the diode 32C, a circuit is established consisting of the antenna 30, the diode 32B, the diode 32C, the resistor 36, and the transducer 34B. An HF voltage at the antenna 30 caused by the HF component 14B will be coupled to the transducer 34B with an out-of-phase phase condition of a 180 degree phase shift relative to transducer 34A. This inversion occurs whether the transducers are being used in a transmit or a receive mode.

Alternatively, during this forward bias condition of the diode 32B and the diode 32C, the external electronics 2 can be controlled to generate no HF component. Consequently, an HF frequency signal such as an ultrasonic signal impinging on the transducer 34B will cause the transducer 34B to produce a current which will subtract from any current from the transducer 34A. The combined difference in current will conduct in the above described circuit to the antenna 30, where it will produce the HF component 14B to be detected by the external electronic system 2.

Fourth Implementation

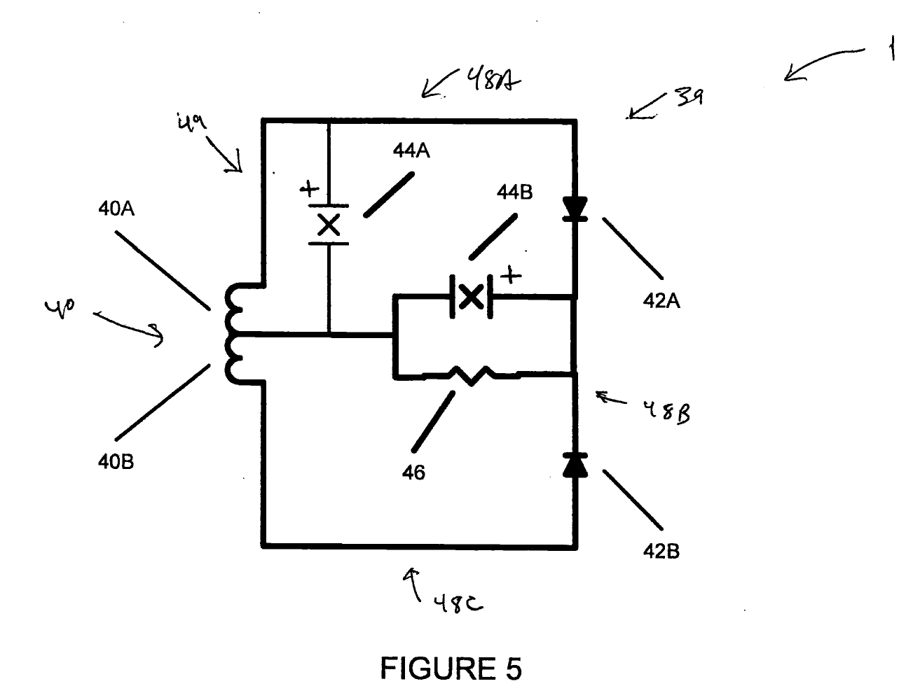

A fourth implementation of the transducer wireless control system 1 has a fourth transducer sub-system 39 shown in FIG. 5 as including an inductive antenna 40 divided into a first inductor portion 40A and a second inductor portion 40B, a first diode 42A, a second diode 42B, a first transducer 44A, a second transducer 44B, and a resistor 46 arranged in a first sub-circuit 48A, a second sub-circuit 48B, and a third sub-circuit 48C. The first sub-circuit 48A includes a combination 49 of the first inductor portion 40A connected in parallel with the first transducer 44A. The first sub-circuit 48A further includes the first diode 42A connected to the combination 49 in series with the first diode oriented for forward biased current to flow away from the combination. The second sub-circuit 48B includes the second transducer 44B connected with the resistor 46 in parallel. The third sub-circuit 48C includes the second inductor portion 40B connected in series with the second diode 42B with the second diode oriented for forward biased current to flow toward the second inductor portion 40B.

The fourth implementation enables the external electronic system 2 to control the transmit and the receive polarities of the first transducer 44A and the second transducer 44B relative to one another. The inductive antenna 40 can be a center-tapped inductor, which is formed by the first inductor portion 40A and the second inductor portion 40B. The inductive antenna 40 is used to receive the HF component 14A, the HF component 14B, and the LF component 18 of the magnetic field 16.

When the LF component 18 produces a voltage on the first inductor portion 40A sufficient to forward bias the first diode 42A, a circuit will be established including the first diode 42A, the first transducer 44A, the second transducer 44B, the resistor 46, and the first inductor portion 40A. The HF voltage present at the first inductor portion 40A will be coupled with the same phase to both the first transducer 44A and the second transducer 44B.

Alternatively, during this forward bias condition of the first diode 42A, the external electronic system 2 can refrain from transmitting the HF component 14A or the HF component 14B. Consequently, an HF frequency signal such as an ultrasonic signal impinging on the second transducer 44B will cause the second transducer to produce a current which will add to any current signal from the first transducer 44A produced by another HF signal impinging thereon. The combined current signal will conduct to the first inductor portion 40A, where it will produce the HF component 14A to be detected by the external electronic system 2.

The voltages induced on the first inductor portion 40A and the second inductor portion 40B, are 180 degrees out of phase with each other. Also, when the first diode 42A is forward-biased (on), the second diode 42B is reverse-biased (off), and vice-versa. Consequently, when the LF component 18 produces a voltage on the second inductor portion 40B sufficient to forward bias the second diode 42B, a circuit will be established including the second diode 42B, the second transducer 44B, the resistor 46, and the second inductor portion 40B. At the same time, the first transducer 44A will form a circuit with the first inductor portion 40A. Thus, the HF voltage present at the second inductor portion 40B will be coupled to the second transducer 44B. The HF voltage present at the first inductor portion 40A will be coupled to the first transducer 44A. As the HF voltages generated at the first inductor portion 40A and the second inductor portion 40B have a phase difference of 180 degrees, the HF voltages 14B presented to the first transducers 44A and the second transducer 44B will have a phase difference of 180 degrees.

Alternatively, during this forward bias condition of diode 42B the external electronic system 2 can refrain from transmitting the HF component 14A and the HF component 14B. Consequently, an HF frequency signal such as an ultrasonic signal impinging on the second transducer 44B will cause the second transducer to produce a current which will be 180 degrees out of phase with any current signal produced from the first transducer 44A. The combined current signal from the first transducer 44A and the second transducer 44B will conduct to the first inductor 40A, where it will produce the HF component 14A to be detected by the external electronic system 2.

As is conventionally known, the phase difference between two RF signals A and B may be found by adding and subtracting the two RF signals. The ratio of the amplitudes of the resultant signals is proportional to the phase angle between them, i.e.,

Phase difference is proportional to |A−B|/|A+B|

By using either the third transducer sub-system 29 found in the third implementation or the fourth transducer sub-system 39 found in the fourth implementation, the phase of one of the two signals can be switched and selection of one of two output signal levels can occur before and after switching. According to the above description, the ratio of the amplitudes of these two signals represents the phase difference. This can have application in measurements of flow using the ultrasonic transit-time technique, which relies upon first transmitting a signal from a first transducer and receiving it at a second transducer, and then reversing the connection to transmit on the second transducer and receive on the first transducer, the transducers being positioned upstream and downstream of a point along a conduit. The phase of the signal traveling in the direction of fluid flow is advanced, while the phase of the signal traveling against the direction of fluid flow is retarded. The flow rate is proportional to the phase difference.

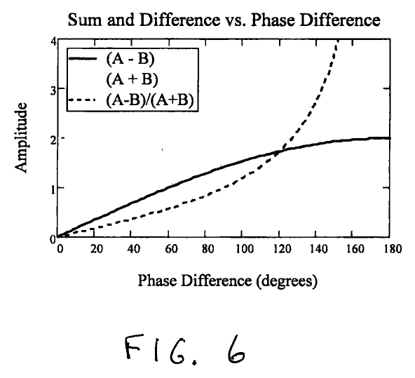

The circuits shown in FIGS. 4 and 5 enable a transmit HF signal to be applied to both transducers simultaneously. The diode bias condition used during transmit may be maintained until midway through the receive HF waveform, and then switched. In this case, the amplitude of the first portion of the receive waveform represents the sum signal (A+B) and the amplitude of the second portion represents the difference signal (A−B). These amplitudes can be used in the equation above to compute the transit-time phase difference. Since the transit-time phase difference is typically quite small (on the order of a few degrees of phase) for flow signals of biological or biomedical interest, reducing the measurement to a simple amplitude ratio simplifies remote measurement via a wireless link. FIG. 6 shows an exemplary graph showing resultant signals associated with both sum and difference versus phase difference.

Comments above regarding selection of diodes and necessity for resistors can be applicable in general to the depicted implementations. From the foregoing it will be appreciated that, although specific embodiments of the invention have been described herein for purposes of illustration, various modifications may be made without deviating from the spirit and scope of the invention. Accordingly, the invention is not limited except as by the appended claims.

The invention claimed is:

1. For implanting into a subject, a system comprising:
an inductive antenna having a first connection portion and a second connection portion;
a first sub-circuit having a first diode and a first transducer portion, the first transducer portion including a first transducer, the first diode and the first transducer portion being connected in series; and
a second sub-circuit having a second diode and a second transducer portion, the second transducer portion including a second transducer, the second diode and the second transducer portion being connected in series, the inductive antenna, the first sub-circuit and the second sub-circuit being connected in parallel, the first diode oriented for forward biased current to flow from the first connection portion toward the inductive antenna, the second diode oriented for forward biased current to flow from the inductive antenna toward the second connection portion.

2. The system of claim 1, the first transducer portion further including a first resistor wherein the first transducer and the first resistor are connected in parallel, and the second transducer portion further including a second resistor wherein the second transducer and the second resistor are connected in parallel.

3. The system of claim 1 wherein the system is sized to be implanted into a vascular portion of the subject.

4. The system of claim 1 wherein the system is sized to be inserted into a location using a tubular structure with a diameter of 0.2 to 6 mm, the tubular structure being one of the following: a catheter, a cannula, and a guidewire.

5. The system of claim 1 wherein the first diode and the second diode have relatively high values for reverse breakdown voltage.

6. The system of claim 1 wherein the first diode and the second diode are PIN junction diodes.

7. The system of claim 1 wherein the first transducer and the second transducer are ultrasonic transducers.

8. For implanting into a subject, a system comprising:
a first sub-circuit having a first transducer and a first diode being connected in parallel;
a second sub-circuit having a second transducer and a second diode being connected in parallel; and
an inductive antenna being connected in series with the first sub-circuit and the second sub-circuit, the first diode oriented for forward biased current to flow from the inductive antenna through the first diode to the second transducer, the second diode oriented for forward biased current to flow from the inductive antenna through the second diode to the first transducer.

9. The system of claim 8, the first sub-circuit further including a resistor wherein the first transducer, the first diode and the first resistor are connected in parallel, and the second sub-circuit further including a second resistor wherein the second transducer, the second diode, and the second resistor are connected in parallel.

10. The system of claim 8 wherein the system is sized to be implanted into a vascular portion of the subject.

11. The system of claim 8 wherein the system is sized to be inserted into a location using a tubular structure with a diameter of 0.2 to 6 mm.

12. The system of claim 11 wherein the tubular structure is one of the following: catheter, cannula, and guidewire.

13. The system of claim 8 wherein the first diode and the second diode have relatively high values for reverse breakdown voltage.

14. The system of claim 8 wherein the first diode and the second diode are PIN junction diodes.

15. The system of claim 8 wherein the first transducer and the second transducer are ultrasonic transducers.

16. A method comprising:
providing an implant with an antenna, a first transducer, and a second transducer;
Implanting the implant into a subject; and
transmitting a magnetic field having a first frequency component to activate the first transducer and deactivate the second transducer when the first frequency component has a first amplitude and to deactivate the first transducer and activate the second transducer when the first frequency component has a second amplitude; and
transmitting the magnetic field with a second frequency component to be received by the antenna in the implant to cause the activated one of the first transducer and the second transducer to transmit a signal having a frequency related to the second frequency component.

17. The method of claim 16 wherein transmitting the first frequency component of the magnetic field activates one of the first transducer and the second transducer through use of a first diode and a second diode.

18. The method of claim 16 wherein implanting positions the implant within a vasculature of the subject.

19. The method of claim 16 wherein the first frequency component is of a lower frequency content than the second frequency component.

20. A method comprising:
providing an implant with an antenna, a first transducer and a second transducer;
implanting the implant into a subject; and
transmitting a magnetic field having a first frequency component to activate the first transducer and deactivate the second transducer when the first frequency component has a first amplitude and to deactivate the first transducer and activate the second transducer when the first frequency component has a second amplitude; and
at a location external to the subject receiving a signal from the antenna in the implant generated by the activated one of the first transducer and the second transducer generated as a result of a signal being received by the activated one of the first transducer and the second transducer.

21. The method of claim 20 wherein implanting positions the implant within a vasculature of the subject.

22. The method of claim 20 wherein the first frequency component is of a lower frequency content than the second frequency component.

23. The method of claim 20 wherein transiting the first frequency component of the magnetic field activates one of the first transducer and the second transducer through use of a first diode and a second diode.

24. For implanting into a subject, a system comprising:
an inductive antenna;
a first transducer; and
a sub-circuit being connected with the inductive antenna and the first transducer in parallel, the sub-circuit having a first component portion, a second component portion, and a third component portion, the first and second component portions being in parallel with the antenna and the first transducer, the first component portion having a first diode and a second diode being connected in series with their anodes in common, the second component portion having a third diode and a fourth diode being connected in series, with their cathodes in common, the third component portion having a second transducer being connected between the anodes of the first component portion and the cathodes of the second component portion.

25. The system of claim 24, the second component portion further including a resistor wherein the second transducer and the resistor are connected in parallel.

26. The system of claim 24 wherein the system is sized to be implanted into a vascular portion of the subject.

27. The system of claim 24 wherein the system is sized to be inserted into a location using with a diameter of 0.2 to 6 mm.

28. The system of claim 27 wherein the tubular structure is one of the following: catheter, cannula, and guidewire.

29. The system of claim 24 wherein the first diode and the second diode have relatively high values for reverse breakdown voltage.

30. The system of claim 24 wherein the first diode and the second diode are PIN junction diodes.

31. The system of claim 24 wherein the first transducer and the second transducer are ultrasonic transducers.

32. For implanting into a subject, a system comprising:
an inductive antenna including a first inductor portion and a second inductor portion;
a first diode;
a first transducer, the first inductor portion and the first transducer being connected in parallel to form a first combination, the first diode being connected in series with the first combination to form a first sub-circuit;
a second sub-circuit including a second transducer; and
a second diode connected in series with the second inductor portion to form a third sub-circuit, the third sub-circuit, the first sub-circuit, and the second sub-circuit being connected in parallel, the first diode and the second diode oriented with their respective forward biased currents flowing toward opposite ends of the inductive antenna.

33. The system of claim 32, the second sub-circuit further including a resistor wherein the second transducer and the resistor are connected in parallel.

34. The system of claim 32 wherein the system is sized to be implanted into a vascular portion of the subject.

35. The system of claim 32 wherein the system is sized to be inserted into a location using a tubular structure with a diameter of 0.2 to 6 mm.

36. The system of claim 35 wherein the tubular structure is one of the following: catheter, cannula, and guidewire.

37. The system of claim 32 wherein the first diode and the second diode have relatively high values for reverse breakdown voltage.

38. The system of claim 32 wherein the first diode and the second diode are PIN junction diodes.

39. The system of claim 32 wherein the first transducer and the second transducer are ultrasonic transducers.

40. A method comprising:
providing an implant with an antenna, a first transducer, and a second transducer;
Implanting the implant into a subject; and
transmitting a magnetic field having a first frequency component to be received by the antenna of the implant to orient a phase condition between the first transducer and the second transducer, as a first phase condition when the first frequency component has a first amplitude and a second phase condition when the first frequency component has a second amplitude; and
transmitting the magnetic field with a second frequency component to be received by the antenna of the implant to cause the first transducer and the second transducer to transmit signals based upon the second frequency component that are in-phase when the phase condition is of the first phase condition and out-of-phase when the phase condition is of the second phase condition.

41. The method of claim 40 wherein implanting positions the implant within a vasculature of the subject.

42. The method of claim 40 wherein the first phase condition is an in-phase condition and the second phase condition is an out-of-phase condition.

43. The method of claim 40 wherein the first frequency component is of lower frequency content than the second frequency component.

44. The method of claim 40 wherein transmitting the first frequency component of the magnetic field orients the phase condition through a first diode and a second diode.

45. A method comprising:
providing an implant with an antenna, a first transducer, and a second transducer;
Implanting the implant into a subject; and
transmitting a magnetic field having a first frequency component to be received by the antenna of the implant to orient a phase condition between the first transducer and the second transducer, as a first phase condition when the first frequency component has a first amplitude and a second phase condition when the first frequency component has a second amplitude; and
at a location external to the subject receiving a signal transmitted from the antenna of the implant that is based upon an addition of a first signal received by the first transducer and a second signal received by second transducer when the phase condition is the first phase condition and based upon a difference of the first signal received by the first transducer and the second signal received by the second transducer when the phase condition is the second phase condition.

46. The method of claim 45 wherein implanting positions the implant within a vasculature of the subject.

47. The method of claim 45 wherein the first phase condition is an in-phase condition and the second phase condition is an out-of-phase condition.

48. The method of claim 45 wherein the first frequency component is of a lower frequency content than the second frequency component.

49. The method of claim 45 wherein transmitting the first frequency component of the magnetic field orients the phase condition through a first diode and a second diode.

50. For implanting into a subject, a system comprising:
an inductive antenna;
first and second diodes; and
first and second transducers configured to perform at least one of transmitting and receiving high frequency signals, the inductive antenna, the first and second diodes and the first and second transducers being so coupled to provide the first and second diodes as switches to direct the high-frequency signals.

51. The system of claim 50 wherein the diodes are biased through low-frequency bias currents.

52. The system of claim 50 wherein the first diode and the first transducer are connected in series as a portion of a first sub-circuit.

53. The system of claim 52 the second diode and the second transducer are connected in series as a portion of a second sub-circuit, the inductive antenna, the first sub-circuit and the second sub-circuit being connected in parallel, the first diode oriented for forward biased current to flow from the first connection portion toward the inductive antenna, the second diode oriented for forward biased current to flow from the inductive antenna toward the second connection portion.

54. The system of claim 53 wherein the first sub-circuit further includes a first resistor wherein the first transducer and the first resistor are connected in parallel and the second sub-circuit further including a second resistor wherein the second transducer and the second resistor are connected in parallel.

55. The system of claim 50 wherein the system is sized to be implanted into a vascular portion of the subject.

56. The system of claim 50 wherein the system is sized to be inserted into a location using a tubular structure with a diameter of 0.2 to 6 mm, the tubular structure being one of the following: a catheter, a cannula, and a guidewire.

57. The system of claim 50 wherein the first diode and the second diode have relatively high values for reverse breakdown voltage.

58. The system of claim 50 wherein the first diode and the second diode are PIN junction diodes.

59. The system of claim 50 wherein the first transducer and the second transducer are ultrasonic transducers.

60. A method comprising:
providing an inductive antenna, first and second diodes; and first and second transducers as at least a portion of an implantable system;
performing at least one of transmitting and receiving high frequency signals with at least one of the first and second transducers; and
biasing the first and second diodes to direct the high-frequency signals through a switching action of at least one of the first and second diodes.

61. The method of claim 60 wherein biasing the diodes is done through low-frequency bias currents.

62. The method of claim 60 wherein the first diode and the first transducer are connected in series as a portion of a first sub-circuit.
